(19) United States
(12) Patent Application Publication
(10) Pub. No.: US 2007/0196300 A1
(43) Pub. Date: Aug. 23, 2007

(54) Multi-phase, emulsion-containing compositions

(75) Inventors: Ria Johns, Fayetteville, NY (US); Xavier Blin, Paris (FR); Michell Chen, Edison, NJ (US); Padraig McDermott, Westfield, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/355,942

(22) Filed: Feb. 17, 2006

(51) Int. Cl. A61K 8/31

(52) U.S. Cl. 424/64; 424/401

(57) ABSTRACT

The invention relates to a composition, especially a cosmetic composition, containing (a) an external oil phase; (b) an internal aqueous phase; and (c) an internal water-in-oil emulsion.

### FIELD OF THE INVENTION

The present invention generally relates to compositions, for example, cosmetic care, treatment or make-up compositions, comprising (a) an external oil phase; (b) an internal aqueous phase; and (c) an internal water-in-oil emulsion. Such compositions possess improved moisturizing and hydration properties.

### DISCUSSION OF THE BACKGROUND

Current cosmetic products such as lipsticks place a strong emphasis on comfort and moisturization. Such products typically achieve moisturization of the keratin materials to which they have been applied by forming a film or barrier on the keratin material to prevent water loss, thereby allowing the keratin material to retain its original water content. To date, however, such cosmetic products have typically been unable to actively moisturize keratin materials. Thus, there remains a need in the art for unique compositions which can provide active moisturization or hydration to keratin materials such as skin or lips.

Accordingly, one aspect of the present invention is a care and/or makeup and/or treatment composition for keratinous material such as the skin and/or the lips which is able to provide improved moisturization or hydration properties to the keratin material, including active moisturization or hydration, upon application.

### SUMMARY OF THE INVENTION

The present invention relates to compositions, preferably cosmetic compositions, comprising (a) an external oil phase; (b) an internal aqueous phase, preferably an internal aqueous gel phase; and (c) an internal water-in-oil emulsion.

The present invention also relates to compositions, preferably cosmetic compositions, comprising (a) an external oil phase; (b) an internal aqueous phase, preferably an internal aqueous gel phase; and (c) an internal water-in-oil emulsion, wherein the internal aqueous phase, the water phase of the water-in-oil emulsion, or both comprises at least one water-soluble active agent such as, for example, a humectant or a moisturizing agent.

The present invention also relates to stick compositions, preferably lipsticks or stick foundations, comprising (a) an external oil phase; (b) an internal aqueous phase, preferably an internal aqueous gel phase; and (c) an internal water-in-oil emulsion, wherein preferably the internal aqueous phase, the water phase of the water-in-oil emulsion, or both comprises at least one water-soluble active agent such as, for example, a humectant or a moisturizing agent.

The present invention also relates to methods of delivering a water-soluble active agent to a keratin material such as skin or lips comprising applying to the keratin material a composition comprising (a) an external oil phase; (b) an internal aqueous phase, preferably an internal aqueous gel phase; and (c) an internal water-in-oil emulsion, wherein the internal aqueous phase, the water phase of the water-in-oil emulsion, or both comprises at least one water-soluble active agent.

The present invention also relates to methods of immediate and sustained delivery of water-soluble active agents such as, for example, humectants or moisturizing agents to keratin materials such as skin or lips comprising applying to the keratin material a composition comprising (a) an external oil phase; (b) an internal aqueous phase, preferably an internal aqueous gel phase; and (c) an internal water-in-oil emulsion, wherein both the internal aqueous phase and the water phase of the water-in-oil emulsion comprise a water-soluble active agent.

The present invention further relates to methods of camouflaging and/or disguising skin or lip imperfections comprising applying a skin or lip imperfection camouflaging and/or disguising effective amount of a composition (a) an external oil phase; (b) an internal aqueous phase, preferably an internal aqueous gel phase; and (c) an internal water-in-oil emulsion, wherein preferably the internal aqueous phase, the water phase of the water-in-oil emulsion, or both comprises at least one water-soluble active agent such as, for example, a humectant or a moisturizing agent to skin or lips in need of such camouflaging or disguising.

The present invention also relates to methods of treating, caring for and/or making up keratinous material (for example, skin or lips) by applying compositions of the present invention to the keratinous material in an amount sufficient to treat, care for and/or make up the keratinous material.

The present invention further relates to methods of enhancing the appearance of keratinous material (for example, skin or lips) by applying compositions of the present invention to the keratinous material in an amount sufficient to enhance the appearance of the keratinous material.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human lips followed by “kissing” a material, for example, a sheet of paper, after expiration of a certain amount of time following application, such as 2 minutes after application. Similarly, transfer resistance of a composition may be evaluated by the amount of product transferred from a wearer to any other substrate, such as transfer from the neck of an individual to a collar after the expiration of a certain amount of time following application. The amount of composition transferred to the substrate (e.g., collar, or paper) may then be evaluated and compared. For example, a composition may be transfer resistant if a majority of the product is left on the wearer, e.g., lips, neck, etc. Further, the amount transferred may be compared with that transferred by other compositions, such as commercially available compositions.

“Long wear” compositions as used herein, refers to compositions where at least one property chosen from consistency, texture, and color remains the same as at the time of application, as viewed by the naked eye, after an extended period of time, such as, for example, 1 hour, 2 hours, and further such as 8 hours. Long wear properties may be evaluated by any method known in the art for evaluating such properties. For example, long wear may be evaluated by a test involving the application of a composition to human skin (including lips) and evaluating the consistency, texture and color of the composition after an extended period of time. For example, the consistency, texture and color of a lip composition may be evaluated immediately following application and these characteristics may then be re-evaluated and compared after an individual has worn the lip composition for a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc.

The cosmetic compositions and methods of the present invention can comprise, consist of, or consist essentially of the essential elements and limitations of the invention described herein, as well as any additional or optional ingredients, components, or limitations described herein or any otherwise useful ingredient found in personal care compositions intended for application to keratin materials.

In accordance with certain aspects of the present invention, the phrase “liquid oil phase” is understood to mean an oil phase, which is liquid at room temperature (25° C.) and atmospheric pressure (760 mmHg), and which comprises one or more fatty substances that are liquid at room temperature, also known as oils, which are compatible with one another.

In accordance with certain aspects of the present invention, the phrase “structured oil phase” is understood to mean that this structured phase does not run between the fingers and/or is at least thickened.,

Where the liquid oil phase is structured, it makes it possible to limit exudation of the oil phase from solid compositions, and furthermore, to limit, after deposition on the skin or the lips, its migration into the wrinkles and fine lines, which is desired for compositions such as a lipstick or an eyeshadow. Significant migration of the liquid oil phase, laden with coloring materials, leads to an unaesthetic effect around the lips or the eyes, which can accentuate the wrinkles and fine lines. This migration is often mentioned by women as being a major defect of conventional lipsticks and eyeshadows. The term “migration” is understood to mean running of the composition deposited on the lips or skin beyond its initial outline.

“Gloss” is essentially related to the nature of the liquid oil phase. Thus, for example, it is possible to reduce the level of waxes and fillers in a lipstick to increase the gloss of the lipstick, but then the migration of the liquid oil phase increases. In other words, the levels of waxes and/or of fillers necessary for preparation of a stick of suitable hardness have been a restricting factor on the gloss of the deposit.

“Tackiness” as used herein refers to measuring the maximum tensile force, Fₘₐₓ, required while separating two surfaces. Depending on the application envisaged and the formulation being designed, the desirable value for Fₘₐₓ may vary. In some embodiments, the substantially non-tacky compositions have a Fₘₐₓ of less than about 4 Newton (N), less than about 1 N, less than about 0.5 N, less than about 0.3 N, less than about 0.2 N or less than 0.1 N. One of ordinary skill in the art can determine the Fₘₐₓ of the composition by, for example, determining the maximum force of traction, measured with an extensiometer of the LLOYD model LR5K type, needed to detach two surfaces.

For example, two 38 mm² surfaces, A and B, which are solid, rigid, inert, and non-absorbing, are mounted on movable mounts, facing each other. The surfaces may be movable either toward or away from each other, or one may move surface A independently from surface B or vice versa. Prior to insertion into the extensiometer, surface A is coated with the composition to be measured, which may be dissolved in a solvent such as aqueous, hydroalcoholic, hydrocarbon, silicone, and alcoholic solvents in a concentration of from about 10 to about 30%, preferably 20%, the surface A is coated in a thickness of from 1 to 10 mil, preferably 1 mil, and the surface is dried for 24 hours at room temperature, e.g., 22 to 25° C., at a relative humidity of about 50%. Once inserted in the extensiometer, surface A is subjected for 20 seconds to a compression force of 3 N against surface B and then subjected for 30 seconds to tensile force at a rate of 20 mm/minute. The amount of force, Fₘₐₓ, needed to obtain initial separation is then noted. A mean Fₘₐₓ is determined by carrying out the procedure with multiple pairs, preferably at least six pairs, of surface A and surface B.

The composition of the present invention may be in any form. For example, it may be a paste, a solid or a cream. The composition of the invention may be transparent or clear, including for example, a composition without pigments. The composition can also be a molded composition or cast as a stick or a dish. The composition in one embodiment is a solid such as a molded stick or a poured stick. The compositions of the present invention may also be in the form of a lip composition such as a lipstick or a liquid lip color, a foundation or a mascara product.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 37° C., 45° C., 50° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

External Oil Phase

According to the present invention, compositions comprising an external oil phase are provided. “External oil phase” means that when the composition is applied to a keratin material, the phase of the composition contacting the keratin material is an oil phase, rather than an aqueous phase. For sake of comparison, the external phase of a water-in-oil emulsion is an oil phase.

The external oil phase comprises at least one oil. Any oils can be used in accordance with the present invention. The oils can be volatile or non-volatile, silicone-based and/or hydrocarbon-based, etc. Thus, for example, the external oil phase may contain, independently or in combination, volatile silicone oils, non-volatile silicone oils, volatile non-silicone oils and non-volatile non-silicone oils.

In one embodiment, the compositions of the present invention are substantially free of silicone oils (i.e., contain less than about 1% of silicone oil). In another embodiment, the compositions are substantially free of non-silicone oils (i.e., contain less than about 1% of non-silicone oil). In another embodiment, the compositions are substantially free of non-volatile oils (i.e., contain less than about 1% of non-volatile oil). In yet another embodiment, the compositions are substantially free of volatile oils (i.e., contain less than about 1% of volatile oil).

According to one embodiment, the external oil phase may contain one or more volatile silicone oils. Examples of such volatile silicone oils include linear or cyclic silicone oils having a viscosity at room temperature less than or equal to 6 cSt and having from 2 to 7 silicone atoms, these silicones being optionally substituted with alkyl or alkoxy groups of 1 to 10 carbon atoms. Suitable oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE I

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

Further, a volatile linear silicone oil may be employed in the compositions of the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

The volatility of the solvents/oils can be determined using the evaporation speed as set forth in U.S. Pat. No. 6,338,839.

Examples of other silicone oils that may be used in the invention include non-volatile linear polydimethylsiloxanes (PDMSs), that are liquid at room temperature; polydimethylsiloxanes comprising alkyl, alkoxy or phenyl groups, which are pendent and/or at the end of a silicone chain, these groups each containing from 2 to 24 carbon atoms; phenylsilicones, for instance phenyl trimethicones, phenyl dimethicones, phenyl trimethylsiloxydiphenylsiloxanes, diphenyl dimethicones, diphenyl methyldiphenyl trisiloxanes and 2-phenylethyl trimethylsiloxysilicates.

According to other preferred embodiments, the external oil phase may contain one or more non-silicone volatile oils and may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

TABLE 2

| | Flash Point |
|---|---|
| Compound | (° C.) |
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

Examples of other non-silicone oils which can be used in the compositions of the present invention include polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including and better still from 7 to 19 carbon atoms, and R₆ represents a branched hydrocarbon-based chain containing from 1 to 40 carbon atoms, including and better still from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol; and
mixtures thereof.

Preferably, the oils present in the external oil phase represent from about 3% to about 60% by weight of the total weight of the composition, more preferably from about 5% to about 40% of the total weight of the composition, and most preferably from about 10% to about 30%, including all ranges and subranges therebetween.

Oil Phase Structuring Agent

According to the present invention, the external oil phase further comprises at least one oil phase structuring agent. The oil phase structuring agent is present in an amount sufficient to structure (that is, thicken and/or increase the viscosity of) the oil phase. In most preferred embodiments, sufficient oil phase structuring agent is present in the external oil phase to form a solid product such as a stick (for example, a lipstick or a stick foundation).

Suitable structuring agents include any agent which is compatible with the external oil phase and which is capable of thickening, solidifying or increasing the viscosity of the external oil phase. Preferred structuring agents include, but are not limited to, polyorganosiloxane-containing polymers, non-silicone-polyamide copolymers, waxes, and mixtures thereof. Examples of these preferred structuring agents can be found in U.S. patent application Ser. No. 11/254,919, filed Oct. 21, 2005, the entire contents of which is hereby incorporated by reference.

Polyorganosiloxane-containing polymers can generally be described as polymers chosen from homopolymers and copolymers, preferably, with a weight-average molecular mass ranging from about 500 to about 2.5×10⁶ or more, comprising at least one moiety comprising: at least one polyorganosiloxane group comprising, preferably, from 1 to about 10,000 organosiloxane units in the chain of the moiety or in the form of a graft, and at least two groups capable of establishing hydrogen interactions are provided.

Non-silicone-polyamide copolymers include but are not limited to those known in the trade as Uniclear or Sylvaclear. These non silicone polyamides have different terminal end groups, such as ester terminated, known as Uniclear 80 or 100, such as amide terminated, known as Sylvaclear A200, and such as polyalkyleneoxy terminated, known as Sylvaclear AF1900 as well as ester terminated polyesteramides. Such non silicone polyamides are available, for instance, from Arizona Chemical Company, Jacksonville, Fla., and are described in U.S. Pat. No. 5,783,657, U.S. Pat. No. 6,402,408, U.S. Pat. No. 6,268,466, U.S. Pat. No. 6,552,160 the entire contents of which are hereby incorporated by reference.

Suitable waxes are those generally used in cosmetics and dermatology; they are, for example, of natural origin, for instance beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methyl-siloxane esters that are solid at 40° C., for example, at above 55° C.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf.

This hardness is measured in one of two ways. A first test for hardness is according to a method of penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within preferred embodiments of the invention.

As is evident, the hardness of the composition according to preferred embodiments of the invention may, for example, be such that the composition is advantageously self-supporting and can disintegrate easily to form a satisfactory deposit on the skin and/or the lips and/or superficial body growths, such as keratinous fibers. In addition, this hardness may impart good impact strength to the inventive compositions, which may be molded or cast, for example, in stick or dish form.

Preferably, the oil phase structuring agent represents from about 1% to about 95% by weight of the total weight of the composition, more preferably from about 1% to about 45% of the total weight of the composition, more preferably from about 3% to about 40% of the total weight of the composition, and most preferably from about 5% to about 30%, including all ranges and subranges therebetween.

Internal Aqueous Phase

According to the present invention, compositions comprising an internal aqueous phase are provided. “Internal aqueous phase” means that when the composition is applied to a keratin material, the phase of the composition contacting the keratin material is not an aqueous phase. For sake of comparison, the internal phase of a water-in-oil emulsion is a water phase.

The internal aqueous phase comprises water. Preferably, the water present in the internal aqueous phase represents from about 1% to about 70% by weight of the total weight of the composition, more preferably from about 5% to about 60% of the total weight of the composition, and most preferably from about 10% to about 50%, including all ranges and subranges therebetween.

Gelling Agent

According to particularly preferred embodiments of the present invention, the internal aqueous phase comprises at least one gelling agent. In most preferred embodiments, sufficient gelling agent is present in the internal aqueous phase to form an internal aqueous gel phase.

Suitable gelling agents include any agent which is compatible with the internal aqueous phase and which is capable of thickening, solidifying or increasing the viscosity of the internal aqueous phase. Suitable gelling agents include but are not limited to polysaccharides such as cellulose derivatives (carboxymethylcellulose, hydroxypropylmethylcellulose); natural gums such as xanthan, guar and carob gums, scleroglucans and chitin or chitosan derivatives; proteins or their hydrolysates such as keratin, gelatin and collagen; and acrylic and methacrylic derivatives such as glycerol polyacrylate (including the product sold by the company SEDERMA under the name “LUBRAJEL®”) and ammonium acrylate copolymer (including the product sold by the company HOECHST under the name “PAS 5161®”); polyethylene glycols (PEG) such as the products sold by the company UNION CARBIDE under the name “CARBOWAX®” Also, compounds identified as “Viscosity Increasing Agents—Aqueous” in the International Cosmetic Ingredient Dictionary and Handbook are examples of suitable gelling agents.

Preferably, the gelling agent, if present, represents from about 0.1% to about 5% by weight of the total weight of the composition, more preferably from about 0.3% to about 4% of the total weight of the composition, and most preferably from about 0.5% to about 3%, including all ranges and subranges therebetween.

Water-Soluble Active Agent

According to particularly preferred embodiments of the present invention, the internal aqueous phase comprises at least one water-soluble active agent. Suitable water-soluble active agents include any agent which is compatible with or soluble in water and which is capable of providing a beneficial effect to keratin materials. Examples of suitable water-soluble active agents generally include but are not limited to antioxidants, free-radical scavengers, moisturizers, humectants, bleaching agents, liporegulators, anti-acne agents, antiseborrhoeic agents, anti-ageing agents, softeners, anti-wrinkle agents, keratolytic agents, anti-inflammatories, refreshing agents, cicatrizing agents, vascular protective agents, antibacterials, antifungals, antiperspirants, deodorants, skin conditioners, desensitizing agents, immunomodulators and nourishing agents which are compatible with or soluble in water.

Specific examples of such water-soluble active agents include but are not limited to ascorbic acid and its biologically compatible salts, enzymes, antibiotics, alpha hydroxy acids and their salts, hydroxylated polyacids, sucrose and its derivatives, urea, amino acids, oligopeptides, water-soluble plant and yeast extracts, protein hydrolysates, hyaluronic acid, mucopolysaccharides, vitamins B₂, B₆, H and PP, panthenol, folic acid, acetylsalicylic acid, allantoin, glycyrrhetic acid, kojic acid and hydroquinone.

Particularly preferred water-soluble active agents are humectants or moisturizing agents such as, for example, polyhydroxy compounds including but not limited to glycerin and glycols such as, for example, propylene glycol, butylene glycol, dipropylene glycol and diethylene glycol, glycol ethers such as monopropylene, dipropylene and tripropylene glycol alkyl(C₁-C₄)ethers, monoethylene, diethylene and triethylene glycol.

Preferably, the water-soluble active agent, if present, represents from about 0.1% to about 40% by weight of the total weight of the composition, more preferably from about 0.5% to about 30% of the total weight of the composition, more preferably from about 1% to about 15% of the total weight of the composition, and most preferably from about 3% to about 10%, including all ranges and subranges therebetween.

When a water-soluble active agent is present in the internal aqueous phase, the internal aqueous phase allows the water-soluble active agent to be immediately delivered to a keratin material upon application of the composition to the keratin material. Thus, for example, when a lipstick comprising an internal aqueous phase containing glycerin and/or a glycol is applied to lips, the water and glycerin/glycol from the internal aqueous phase are directly delivered to the lips, thereby providing relatively instantaneous hydration to the lips. Similarly, when a foundation comprising an internal aqueous phase containing ascorbic acid is applied to skin, the water and ascorbic acid from the internal aqueous phase are delivered directly to skin, thereby providing relatively instantaneous antioxidant activity (among other activities) to the skin.

Water-in-Oil Emulsion

According to the present invention, compositions comprising an internal water-in-oil emulsion are provided. “Internal water-in-oil emulsion” means that when the composition is applied to a keratin material, the water-in-oil emulsion is not contacting the keratin material. The internal emulsion can contain droplets of any size. However, it is to be understood that, as with any emulsion, smaller and more homogeneous (that is, less diversity of droplet sizes) droplet sizes are preferred because emulsions containing such droplets tend to be more stable. Any suitable means for preparing emulsions can be used to prepare the internal water-in-oil emulsions of the present invention.

According to particularly preferred embodiments of the present invention, the water phase of the water-in-oil emulsion comprises at least one water-soluble active agent. Suitable water-soluble active agents and the amounts thereof in the water phase of the emulsion are the same as discussed above in connection with the internal aqueous phase.

In a particularly preferred embodiment, both the internal aqueous phase and the water phase of the water-in-oil emulsion comprise at least one water-soluble active agent. In this particularly preferred embodiment, immediate and sustained delivery of water-soluble active agent can be provided to keratin material.

While not wishing to be bound by any particular theory, it is believed that the water-soluble active agent in the internal aqueous phase is delivered to the keratin material relatively immediately after application and that the keratin material quickly absorbs this water-soluble active agent. Over time, the internal water-in-oil emulsion slowly breaks, thereby releasing the water-soluble active agent in the water phase of the emulsion to the keratin material over time for a sustained delivery of active agent to the keratin material. After the emulsion breaks, the oil phase from this emulsion remains on the keratin material, providing shine and comfort to the keratin material.

It is further believed that the oil phase structuring agent can, upon application of the composition, form a film or barrier which prevents moisture from evaporating from the keratin material, thereby enhancing the effect or activity of the water-soluble active agent.

In particularly preferred embodiments of the present invention, the water-in-oil emulsion is prepared first. Such preparation can occur through any suitable means or methods for preparing emulsions. The internal aqueous phase is then prepared. After these two phases have been prepared, the water-in-oil emulsion is incorporated into the internal aqueous phase to prepare a product containing both internal phases. Finally, this product is incorporated into an external oil phase to yield compositions of the present invention.

Alternatively, after the water-in-oil emulsion and internal aqueous phase have been prepared, they can be added to the external oil phase simultaneously or sequentially without first adding the water-in-oil emulsion to the aqueous oil phase.

Coloring Agents

According to the present invention, the compositions may optionally comprise at least one coloring agent. Suitable coloring agents include but are not limited to pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%. In the case of certain products, the pigments, including nacreous pigments, may, for example, represent up to 50% by weight of the composition.

Additional Additives

The compositions of the present invention can also comprise any additive usually used in the field under consideration. For example, non-encapsulated pigments, film forming agents, dispersants, antioxidants, essential oils, preserving agents, fragrances, liposoluble polymers that are dispersible in the medium, fillers, neutralizing agents, cosmetic and dermatological oil-soluble active agents such as, for example, emollients, moisturizers, vitamins, anti-wrinkle agents, essential fatty acids, sunscreens, and mixtures thereof can be added. A non-exhaustive listing of such ingredients can be found in U.S. patent application Ser. No. 10/733,467, filed Dec. 12, 2003, the entire contents of which is hereby incorporated by reference. Further examples of suitable additional components can be found in the other references which have been incorporated by reference in this application, including but not limited to the applications from which this application claims priority. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present).

Needless to say, the composition of the invention should be cosmetically or dermatologically acceptable, i.e., it should contain a non-toxic physiologically acceptable medium and should be able to be applied to the keratin material (such as skin, superficial body growths or the lips) of human beings. For the purposes of the invention, the expression “cosmetically acceptable” means a composition of pleasant appearance, odor, feel and/or taste.

According to preferred embodiments of the present invention, methods of treating, caring for and/or making up keratinous material such as skin, lips, hair and mucous membranes by applying compositions of the present invention to the keratinous material in an amount sufficient to treat, care for and/or make up the keratinous material are provided.

According to other preferred embodiments, methods of covering or hiding defects associated with keratinous material such as imperfections or discolorations by applying compositions of the present invention to the keratinous material in an amount sufficient to cover or hide such defects are provided.

According to yet other preferred embodiments, methods of enhancing the appearance of keratinous material by applying compositions of the present invention to the keratinous material in an amount sufficient to enhance the appearance of the keratinous material are provided.

According to preferred embodiments of the present invention, methods for delivering a water-soluble active agent to keratin materials such as skin or lips comprising applying to the keratin material a composition comprising (a) an external oil phase; (b) an internal aqueous phase; and (c) an internal water-in-oil emulsion comprising, in the water phase, a water-soluble active agent are provided.

According to other preferred embodiments, methods for immediate and sustained delivery of water-soluble active agents such as humectants or moisturizing agents to keratin materials such as skin or lips comprising applying to the skin or lips a composition comprising (a) an external oil phase; (b) an internal aqueous phase comprising at least one humectant or moisturizing agent; and (c) an internal water-in-oil emulsion comprising, in the water phase, at least one humectant or moisturizing agent are provided.

In accordance with the preceding preferred embodiments, the compositions of the present invention are applied topically to the desired area of the skin in an amount sufficient to treat, care for and/or make up the keratinous material, to cover or hide defects associated with keratinous material, skin imperfections or discolorations, or to enhance the appearance of keratinous material. The compositions may be applied to the desired area as needed, preferably once or twice daily, more preferably once daily and then preferably allowed to dry before subjecting to contact such as with clothing or other objects. The composition is preferably applied to the desired area that is dry or has been dried prior to application.

The present invention also envisages kits and/or prepackaged materials suitable for consumer use containing one or more compositions according to the description herein. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLE 1

### Lipstick

A. Preparation of Water-in-Oil Emulsion:

Oil Phase:

The following ingredients were added into a manufacturing tank:

| Amount (g) | Ingredient |
|---|---|
| 0.8 | Sodium Tristearate |
| 4.02 | Cetyl Alcohol |
| 0.67 | Potassium Cetyl Phosphate |
| 4.02 | Hydrogenated Polyisobutene |
| 5.62 | Diisostearyl Malate |
| 4.02 | Dimethicone |
| 12.05 | PEG-45 Dodecyl Glycol Copolymer (ELFACOS |
| | ST 9 from AKZO NOBEL) |

The mix was heated to 80° C. to complete a good melting of the waxes and solid surfactants. The aqueous phase (see below) was heated to 75° C. and was then slowly added to the mix with a rotor-stator homogenizer (Silverson L4RT-A from Silverson Machines, Ltd., Manchester, England) at about 7200 rpm for 15 min.

Aqueous Phase:

| Amount (g) | Ingredient |
|---|---|
| 43.63 | Water |
| 0.6 | Preservatives |
| 1.78 | PEG-40 Stearate |

The mix was then passed through the homogenizer three times. The resulting emulsion was a water-in-oil emulsion.

Aqueous Gel Phase:

| Amount(g) | Ingredient |
|---|---|
| 7.5 | Glycerin |
| 3.0 | Pentylene Glycol |
| 8.66 | Water |
| 0.5 | Steareth-100/PEG-136/HDMI Copolymer |
| | (NUVIX FX 100 available from Elementis) |

The water and the associative gellant (Steareth-100/PEG-136/HDMI Copolymer) were mixed together in a stirred tank at room temperature in order to develop the gellant for at least 60 mn.

The glycerin and pentylene glycol were then added under mixing conditions. Thus, an aqueous gel product was formed.

The water-in-oil emulsion was then incorporated into the aqueous gel phase from the top of the manufacturing tank stirred only with impeller and scraper, and was brought to room temperature.

Lipstick Preparation:

5.7 g Hydrogenated Jojoba Oil was melted at 70° C.

71.3 g of the water-in-oil emulsion/aqueous phase gel product combination was slowly added to the melted Hydrogenated Jojoba Oil with stirring.

When the water-in-oil emulsion/aqueous phase gel product was incorporated into the wax, 21.2 g Diisostearyl malate 71295 was added with stirring and the mix was heated to 65° C.

The mix was poured at 65° C. into a slimline mold and cooled in the freezer for 45 min to form a lipstick product.

What is claimed is:

1. A composition comprising:
(a) an external oil phase comprising at least one oil and at least one oil phase structuring agent;
(b) an internal aqueous phase; and
(c) an internal water-in-oil emulsion.

2. The composition according to claim 1, wherein the oil phase structuring agent comprises at least one wax.

3. The composition according to claim 2, wherein the at least one wax is present in an amount ranging from about 1% to about 95% by weight of the entire weight of the composition.

4. The composition according to claim 2, wherein the at least one wax is present in an amount ranging from about 1% to about 45% by weight of the entire weight of the composition.

5. The composition according to claim 1, wherein the composition is in the form of a stick.

6. The composition according to claim 1, wherein the aqueous phase comprises at least one gelling agent.

7. The composition according to claim 1, wherein the aqueous phase comprises at least one water-soluble active agent.

8. The composition according to claim 7, wherein the water-soluble active agent is a humectant or moisturizing agent.

9. The composition according to claim 7, wherein the water-soluble active agent is glycerin or a glycol.

10. The composition according to claim 8, wherein the at least one humectant or moisturizing agent is present in an amount ranging from about 0.5% to about 30% by weight of the entire weight of the composition.

11. The composition according to claim 8, wherein the at least one humectant or moisturizing agent is present in an amount ranging from about 1% to about 15% by weight of the entire weight of the composition.

12. The composition according to claim 1, wherein the water-in-oil emulsion comprises, in the water phase, a water-soluble active agent.

13. The composition according to claim 12, wherein the water-soluble active agent is a humectant or moisturizing agent.

14. The composition according to claim 1, wherein the composition is in the form of a lipstick.

15. The lipstick according to claim 14, wherein the oil phase structuring agent comprises at least one wax.

16. The lipstick according to claim 15, wherein the at least one wax is present in an amount ranging from about 1% to about 95% by weight of the entire weight of the composition.

17. The lipstick according to claim 15, wherein the at least one wax is present in an amount ranging from about 1% to about 45% by weight of the entire weight of the composition.

18. The lipstick according to claim 14, wherein the aqueous phase comprises at least one gelling agent.

19. The lipstick according to claim 14, wherein the aqueous phase comprises at least one water-soluble active agent.

20. The lipstick according to claim 19, wherein the water-soluble active agent is a humectant or moisturizing agent.

21. The lipstick according to claim 19, wherein the water-soluble active agent is glycerin or a glycol.

22. The lipstick according to claim 20, wherein the at least one humectant or moisturizing agent is present in an amount ranging from about 0.5% to about 30% by weight of the entire weight of the composition.

23. The lipstick according to claim 20, wherein the at least one humectant or moisturizing agent is present in an amount ranging from about 1% to about 15% by weight of the entire weight of the composition.

24. The lipstick according to claim 14, wherein the water-in-oil emulsion comprises, in the water phase, a water-soluble active agent.

25. The lipstick according to claim 20, wherein the water-in-oil emulsion comprises, in the water phase, a humectant or moisturizing agent.

26. A method for immediate and sustained delivery of humectants or moisturizing agents to lips comprising applying to the lips a composition according to claim 25.
